(19) United States
(12) Patent Application Publication
(10) Pub. No.: US 2026/0253488 A1
(43) Pub. Date: Aug. 27, 2026

(54) System, Gas Measuring Device and Method for Changing a Safety-Relevant Configuration Value

(71) Applicant: Dräger Safety AG & Co. KGaA, Lübeck (DE)

(72) Inventors: Jun Furuta, Lübeck (DE); Tim Gnoerrlich, Lübeck (DE)

(21) Appl. No.: 19/548,322

(22) Filed: Feb. 24, 2026

(30) Foreign Application Priority Data
Feb. 25, 2025 (DE) 102025107085.7

(51) Int. Cl. G08C 17/02; G01N 33/00

(52) U.S. Cl. CPC G08C 17/02; G01N 33/0027

(57) ABSTRACT

The present disclosure relates to a system for changing a safety-relevant configuration value of a gas measuring device, comprising the gas measuring device and an operating unit that is connected to the gas measuring device for bidirectional data transmission, wherein the operating unit is designed to send a configuration value to the gas measuring device. The present disclosure is characterized in that the gas measuring device is designed to generate a request message for manual input confirming the configuration value and send it to the operating unit, furthermore to receive a response message with information about the manual input from the operating unit, and furthermore to compare the information about the manual input with an expected value and generate an information signal and/or switch to a safe state if the information about the manual input deviates from the expected value. Furthermore, the present disclosure relates to a gas measuring device and to a method for changing a safety-relevant configuration value of a gas measuring device.

### CROSS-REFERENCE TO RELATED APPLICATIONS

This application claims the priority of German Patent Application No. 102025107085.7, filed on Feb. 25, 2025, and titled “SYSTEM, GAS MEASURING DEVICE AND METHOD FOR CHANGING A SAFETY-RELEVANT CONFIGURATION VALUE” which is hereby incorporated by reference in its entirety for all nonlimiting purposes.

### TECHNICAL FIELD

The present disclosure relates to a system and to a method for changing a safety-relevant configuration value of a gas measuring device. Furthermore, the invention relates to a gas measuring device for changing a safety-relevant configuration value.

### BACKGROUND

Gas measuring devices typically have an interface for exchanging data with other electronic devices. In particular, measurement data are transmitted. Furthermore, the interface is used to configure the gas measuring device, in particular to change the settings or configuration values of the gas measuring device in order to adapt the function of the gas measuring device to specific circumstances, for example.

In practice, the interfaces for exchanging data with gas measuring devices are often open and/or standardized interfaces, with the interfaces being designed for wireless and/or wired data exchange. An example of an open interface, in particular in the field of stationary gas measuring devices, is the Highway Addressable Remote Transducer (HART) protocol, which allows easy-to-use and standardized communication. Other interfaces are based on Bluetooth, WiFi, or Zigbee, for example.

A key task of gas measuring devices is to warn of dangers to people and/or equipment. Therefore, high demands are placed on gas measuring devices functioning reliably, in particular in terms of functional safety. Furthermore, gas measuring devices that are to be approved or have been approved in accordance with a specific standard, such as EN 50271 or IEC 61508, must meet high requirements regarding their safety and/or reliability.

One aspect for the safe use of a gas measuring device is that it can be or is ensured that a configuration change, in particular a change to the settings of the gas measuring device or a configuration value for a safety function thereof, is carried out in a controlled and reliable manner. In particular, accidentally or unintentionally carrying out such a change is to be avoided, for example by an error in an operating unit that is used for changing the configuration of the gas measuring device and is connected to the gas measuring device to exchange therewith, or by a malware that is implemented on such an operating unit and/or has been transmitted to the gas measuring device.

In practice, it is known to allow a controlled configuration change to a gas measuring device by entering a password. In addition to entering a password, other authentication methods are also possible, for example via digital certificates, electronic signatures, smart cards or tokens. Typically, a password is first entered on an operating unit connected to the gas measuring device for exchanging data therewith, and is then transmitted to the gas measuring device and verified. If the password is correct, a user of the operating unit and the gas measuring device will be able to make a configuration change. The option to configure the gas measuring device is therefore enabled. The user can then make a configuration change using the operating unit.

Typically, the password must be entered again to confirm the configuration change. Authentication methods available in practice often do not support password entry. Furthermore, it is necessary to enter the password again to prevent any further configuration changes. The ability to make a configuration change is therefore first enabled by entering a password and then locked again by entering the password again.

The aforementioned technical solution involves a relatively high level of effort, as, for example, the password has to be entered multiple times. Furthermore, the user must know the password, and the password may need to be synchronized and managed across a plurality of gas measuring devices, which requires suitable data exchange infrastructure.

Another disadvantage is that, for example, the user may forget to re-enter the password to lock the gas measuring device before further configuration changes are made. In this case, the probability increases of an uncontrolled configuration change being carried out, for example by malware, since the gas measuring device has enabled a configuration change by the initial entry of the password and thus allows further configuration changes.

Furthermore, it is conceivable that malware may bypass the protection mechanism for enabling a configuration change, in particular with simple passwords, and make an uncontrolled and potentially dangerous change to a configuration value of the gas measuring device.

### SUMMARY

The object addressed by the present disclosure is that of providing a system and a gas measuring device as well as a method for changing a safety-relevant configuration value in order to increase the safety of a user of the gas measuring device. In particular, any change to the configuration of the gas measuring device is to be reliable, controlled and achievable with little effort. Furthermore, the possibility of malware changing a configuration value of the gas measuring device is to be avoided.

The above problem is solved by a system for changing a safety-relevant configuration value of a gas measuring device having the features of, and by a method according to, the claims. Further details of the present disclosure emerge from the claims, the description and the drawings. In this case, features and details which are described in connection with the system according to the present disclosure also apply in connection with the gas measuring device according to the present disclosure and the method according to the present disclosure so that the disclosure of the individual aspects of the present disclosure always refers, or rather can always refer, to either or.

The system according to the present disclosure for changing a safety-relevant configuration value of a gas measuring device comprises the gas measuring device and an operating unit that is connected to the gas measuring device for bidirectional data transmission. The operating unit is configured to send a configuration value to the gas measuring device. The gas measuring device is configured to detect at least one property of a gas and/or a gas mixture in a monitoring region of the gas measuring device, to measure a concentration each time and/or to compare it with a limit value in order to assess the risk to a person in the monitoring region. Furthermore, the gas measuring device is configured to receive the configuration value from the operating unit and store it. The system is characterized in that the gas measuring device is further configured to generate a request message for manual input confirming the configuration value and send it to the operating unit, the request message containing information about a task to be solved. The request message is also to be understood as a prompt message, whereby these messages send a request or a prompt for manual input. Furthermore, the gas measuring device is configured to receive a response message with information about the manual input from the operating unit, and furthermore to use a comparison between the information about the manual input and an expected value as a basis for generating an information signal and/or switching to a safe state. Furthermore, the operating unit is configured to receive the request message from the gas measuring device and to output at least part of it, and furthermore to generate the response message on the basis of a manual input from a user and send it to the gas measuring device.

The term “gas measuring device” is to be understood to mean both mobile and stationary gas measuring devices. Mobile gas measuring devices are typically designed to be used as easily as possible, in particular without any assembly effort required to fasten or remove the gas measuring device at different locations, whereas stationary gas measuring devices are typically designed for longer term use in a single location and are in particular fastened to a wall, a hall ceiling, a mast or the like.

The gas measuring device comprises in particular at least one gas sensor and/or a communication unit for transmitting gas sensor values and for exchanging data with other devices, such as an operating unit. Furthermore, the gas measuring device comprises a data processing computing unit, for example a microprocessor, and for example a data memory in the form of a semiconductor memory.

A safety-relevant configuration value of a gas measuring device is understood to mean configuration values that have a direct or indirect influence on the safety function of the gas measuring device. The safety function of the gas measuring device comprises, in particular, warning of an ambient atmosphere that is harmful and/or hazardous to health. Safety-relevant configuration values of a gas measuring device comprise, in particular, alarm settings, such as a warning threshold, an alarm threshold and/or an alarm type, apparatus mode settings, such as a measuring mode and/or an adjustment mode, as well as sensor settings, such as an adjustment gas concentration, a gas selected for measurement, a measuring region and/or a maintenance interval. The system, the gas measuring device and the method are designed for changing a single configuration value and/or a plurality of safety-relevant configuration values.

An operating unit is to be understood to mean a device on which a user can carry out manual input. The operating unit comprises, for example, a display unit in the form of a screen, in particular a touch-sensitive screen, an audio unit in the form of a speaker and/or a microphone, a keyboard, a mouse, a biometric scanner and/or a switch. Other input and output devices are conceivable. The operating unit is in particular designed for manual input by a user.

The operating unit is designed, for example, as a computer terminal, a hand-held device, a tablet, or a smartphone. Other apparatuses of a similar type are conceivable.

The gas measuring device and the operating unit are connected to one another for exchanging data therebetween and each has in particular a suitable communication unit. Data exchange takes place via a communication interface designed for wireless and/or wired data transmission.

The gas measuring device, in particular the communication unit and/or computing unit of the gas measuring device, is configured to receive the configuration value and, for example, to store it in a data memory of the gas measuring device.

Furthermore, the gas measuring device is configured to apply the configuration value immediately after receiving the configuration value from the operating unit, or to apply the configuration value immediately after comparing the information about the manual input with the expected value, provided that the information about the manual input substantially matches the expected value.

The application of the configuration value by the gas measuring device is to be understood to mean that the gas measuring device is configured to use the received or stored configuration value, whereby the function of the gas measuring device at least partially changes. If the configuration value is, for example, a new alarm threshold for a specific gas, the gas measuring device compares the measured gas concentration after applying the new alarm threshold with this new alarm threshold, i.e., the configuration value received from the operating unit.

After receiving the configuration value, the gas measuring device is further configured to generate the request message for manual input confirming the configuration value and send it to the operating unit. Confirming the configuration value is in particular intended to make the user aware of the configuration change and to check they agree with it. Preferably, they have the option to check the configuration change, for example by having the changed configuration value displayed to them on the operating unit and being able to compare it with their previous entry. It is conceivable that the configuration value could alternatively or additionally be displayed on a separate display unit, i.e., disconnected from the operating unit.

Confirmation of the configuration value requires user interaction with the operating unit, the type of interaction being determined by the request message and the information about a task to be solved. The request message comprises, in particular, information about a request, instruction, prompt and/or task that can be displayed or executed by the operating unit and with which it can be verified and/or ensured that a person is confirming this request message. The information comprised in the request message is preferably generated by the gas measuring device.

It is essential that the confirmation of the request message by manual input on the operating unit can, with at least high probability, only be carried out by a human. Within the context of the present disclosure, such confirmation can in particular only be carried out by a human being if confirmation by a computer or a machine is not possible or is only possible with considerable effort, the effort being measured, for example, in terms of costs, time and/or technical means. For example, the information in the request message can be generated in accordance with a prompt-response method and/or comprises a puzzle that the user has to solve. The request message does not, for example, comprise a simple request in the form of a confirmation query, where a user simply agrees or disagrees with a configuration change by answering yes or no. Furthermore, it is conceivable that the request message comprises the previously saved configuration value and that this value can be displayed on the operating unit.

The operating unit is further configured to receive the request message from the gas measuring device and to output at least some of the information it contains, wherein a user of the operating unit is given the option and/or prompted to provide manual input on the operating unit in accordance with the request message, for example. As previously described, the operating unit comprises technical means that allow manual input.

The user's manual input can be identified by the operating unit. The operating unit is further configured to use the manual input from the user as a basis for generating the response message, which comprises information about the manual input, and to send it to the gas measuring device. The information about manual input therefore comprises the user's solution to the task they have to solve in the request message.

The gas measuring device is further configured to receive the response message from the operating unit and to check the information about the manual input from the user that it contains. This involves comparing the information about the manual input from the user with at least one expected value. It is conceivable that the expected value is predetermined and stored in the gas measuring device and/or that the expected value can be generated by the gas measuring device on the basis of the request message, in particular on the information about a task to be solved.

If the information about the manual input completely or partially deviates from the expected value, or if no response message containing information about manual input is received, it can be assumed that the configuration change or the manual input on the operating unit was not carried out by a human.

The gas measuring device is further configured to generate an information signal and/or switch to the safe state on the basis of the comparison between the information about the manual input and the expected value. The information signal comprises, in particular, an indication of a detected deviation or an indication that no response message was received. For example, the information signal is a warning signal, an alarm signal and/or an electronic message. The safe state of the gas measuring device depends on the particular protective function that the gas measuring device is required to fulfill. For example, the gas measuring device switches to a special state to indicate that it is not operational. Furthermore, it is conceivable that the gas measuring device might generate an alarm or switch to an error state.

If the information about the manual input substantially matches the at least one expected value, the previously stored configuration value is applied by the gas measuring device, or it remains applied if the configuration value has already been applied previously, in particular after the gas measuring device has received the configuration value.

A particular advantage of the system according to the present disclosure lies in the fact that it ensures that a change to a safety-relevant configuration value can only be carried out or confirmed by a human being and not by a computer, a machine or any other electronic unit. Furthermore, the present disclosure ensures that it is the user who intends to make the change to the configuration value. This allows safe and reliable operation of the gas measuring device.

Another advantage is that the configuration value received by the gas measuring device can be read out and thus verified so that incorrect transmission of the configuration value from the operating unit to the gas measuring device can at least be identified and thus corrected if necessary.

Furthermore, the system according to the present disclosure is designed such that confirming a change to a configuration value is not dependent on authentication, whereby authentication is usually carried out by entering a password. Thus, the authentication method can be flexibly adapted to the circumstances and the means of communication used. A particular advantage is that a change in the authentication method, for example because higher requirements are placed thereon, can be easily made without affecting the confirmation of the change to a configuration value, thus advantageously saving effort and costs.

Further features, objects and effects of the present disclosure will become apparent from the following description of specific exemplary embodiments and from the accompanying figures. Exemplary embodiments of the present disclosure are described without limiting the general inventive concept.

In an example embodiment of the system, the request message differs from a previous request message, wherein the information about a task to be solved that is comprised in the request messages differs. The gas measuring device thus generates an individual request message for each received configuration value or for a plurality of received configuration values, and the individual request messages contain different pieces of information about a task to be solved. The different pieces of information about a task to be solved are either completely different or partially different. For example, the tasks to be solved are different types of tasks, or the tasks to be solved are the same type of tasks, but expect a solution that differs from the solution of the previous task.

A particular advantage of request messages that differ in terms of the information about a task to be solved is that the probability that only a human being and not a computer or machine can solve the tasks is even higher. This makes changing the configuration values of a gas measuring device even safer and more reliable.

In accordance with an example embodiment of the system, the gas measuring device is further configured to generate an information signal and/or switch to a safe state if a predetermined time or predetermined time period between receiving the configuration value and then receiving the response message is exceeded, or if, within the predetermined time or predetermined time period, the information about the manual input does not match the expected value. The predetermined time is preferably stored in a data memory of the gas measuring device. Preferably, the predetermined time or predetermined time period ranges from 5 to 20 minutes, particularly preferably the predetermined time or predetermined time period is 15 minutes. Depending on the application, different predetermined times or time periods are also conceivable. It is conceivable that several configuration values will be changed by the user within the predetermined time. In this case, it is sufficient if a response message was received within the predetermined time or predetermined time period, wherein the information about the manual input substantially matches the expected value.

The gas measuring device and/or, in particular, its computing unit is configured to carry out a time measurement or to allow the predetermined time to elapse. The point in time from which the time measurement starts or the predetermined time elapses is, for example, when the configuration value is received, in particular a first configuration value, when the request message is sent or when the configuration value is applied by the gas measuring device, wherein the configuration value is applied before the response message is received.

The gas measuring device is configured to generate an information signal and/or switch to a safe state if, from one of the aforementioned points in time and within the predetermined time or predetermined time period, it does not receive a response message or a response message related to the request message from the operating unit, or if the information about the manual input in the response message does not match the expected value. The information signal preferably comprises an indication that no response message has been received or that the manual input is incorrect. For example, the information signal is a warning signal, an alarm signal and/or an electronic message. The safe state of the gas measuring device depends on the particular protective function that the gas measuring device is required to fulfill. For example, the gas measuring device switches to a special state to indicate that it is not operational. Furthermore, it is conceivable that the gas measuring device might generate an alarm or switch to an error state.

In an advantageous way, it is at least apparent that the change to the configuration value was not confirmed by the user within the predetermined time or predetermined time period, or that no confirmation took place. This increases safety, as it draws attention to this potentially dangerous condition and/or switches the gas measuring device to a safe state.

In an example embodiment of the system, the request message comprises a fully automated public Turing test for distinguishing between computers and humans (CAPTCHA).

The CAPTCHA preferably comprises image data and/or audio data that can be displayed and/or output by the operating unit. Furthermore, the CAPTCHA preferably comprises a task to be solved. For example, the CAPTCHA comprises an image with graphically distorted text and the task of identifying and/or decoding this text.

The gas measuring device is preferably configured to generate the CAPTCHA. Furthermore, the gas measuring device is configured to compare the information about the manual input from the user with an expected value, where the expected value comprises the correct solution to the CAPTCHA. The expected value can be generated by the gas measuring device, stored in a data memory of the gas measuring device and/or received by the gas measuring device.

For example, the gas measuring device is configured to compare text entered by the user, which was displayed as a distorted image on the operating unit in the form of a CAPTCHA, with the expected value for this CAPTCHA. The entered text is comprised in the information about the manual input in the response message, and the expected value comprises the text that is distorted in the image. If the two texts substantially match, the gas measuring device is configured to apply or continue applying the changed configuration value. If the entered text deviates from the text of the expected value, the gas measuring device switches to the safe state and/or generates an information signal.

In an example embodiment of the system, the bidirectional data transmission between the gas measuring device and the operating unit is based on a Highway Addressable Remote Transducer (HART) communication protocol, an Advanced Physical Layer (APL) standard, or Bluetooth. The system is advantageously suitable for various communication protocols and can be flexibly adapted to the circumstances of where it is used.

In accordance with an example embodiment of the system, the operating unit is configured to authenticate a user and to send the configuration value to the gas measuring device only after the user has been successfully authenticated. The authentication serves in particular to establish the identity of the user and then check whether the user is authorized to change a configuration value of the gas measuring device. In particular, the authentication does not meet the requirement of determining whether the user is a human being.

Authentication is given, for example, by entering a username or password, by means of an electronic signature or is certificate-based authentication. Other methods are conceivable. Furthermore, it is conceivable that several authentication methods are used in the form of multi-factor authentication.

The aforementioned embodiment of the system is advantageously suitable for initially determining whether a user is authorized to make a configuration change and then checking whether the configuration change was made by a human. A particular advantage is that the methods required for this are independent of one another and do not influence one another. For example, it is easy to change the authentication method without affecting the method used to identify whether the user is a human being. This advantageously makes it possible to switch the authentication method to a newer and/or more up-to-date method both quickly and easily.

In an example embodiment of the system, the gas measuring device possesses the properties required in the standard IEC 61508 and is thus designed as a functional safety electronic device. Preferably, the operating unit does not possess the properties required in the standard IEC 61508 or is not approved according to the standard IEC 61508.

IEC 61508 and EN 61508 are identical in content and are to be used synonymously for the purposes of the present disclosure. IEC 61508 concerns the development of electrical, electronic and programmable electronic systems that perform a safety function.

IEC 61508 sets high demands thereon. This is in particular also true if changes are made to the gas measuring device, for example in the form of a software update. Each of the changes may involve significant development and/or documentation effort.

Due to the separation of user authentication and the verification of whether the user is a human being, as previously described, authentication can in particular be advantageously adapted to current methods without a large amount of effort with regard to IEC 61508.

Furthermore, the present disclosure relates to a gas measuring device for changing a safety-relevant configuration value. The gas measuring device is configured to detect a gas and/or a gas mixture in a monitoring region of the gas measuring device, to receive a configuration value from an operating unit and to store the configuration value. The gas measuring device is further configured to generate a request message for manual input confirming the configuration value and send it to the operating unit, the request message containing information about a task to be solved. Furthermore, the gas measuring device is configured to receive a response message with information about the manual input from the operating unit, and furthermore to use a comparison between the information about the manual input and an expected value as a basis for generating an information signal and/or switching to a safe state.

The gas measuring device can be either a mobile gas measuring device or a stationary gas measuring device. The gas measuring device comprises in particular at least one gas sensor and/or a communication unit for transmitting gas sensor values and for exchanging data with other devices, such as the operating unit. Furthermore, the gas measuring device comprises a data processing computing unit, for example a microprocessor, and for example a data memory in the form of a semiconductor memory.

The operating unit is in particular a device on which a user can provide manual input, wherein the operating unit has appropriate technical means, for example a touch-sensitive screen and/or a keyboard.

The gas measuring device has a communication unit for exchanging data with the operating unit, wherein data is transmitted via a communication interface designed for wireless and/or wired data transmission.

The gas measuring device, in particular the communication unit and/or computing unit of the gas measuring device, is configured to receive the configuration value and, for example, to store it in a data memory of the gas measuring device.

Furthermore, the gas measuring device is configured to apply the configuration value immediately after receiving the configuration value from the operating unit, or to apply the configuration value immediately after comparing the information about the manual input with the expected value, provided that the information about the manual input substantially matches the expected value.

After receiving the configuration value, the gas measuring device is further configured to generate the request message for manual input confirming the configuration value and send it to the operating unit. The request message comprises, in particular, information about a request, instruction, prompt and/or task that can be displayed or executed by the operating unit and with which it can be verified and/or ensured that a person is confirming this request message. The information comprised in the request message is preferably generated by the gas measuring device.

The confirmation of the request message by manual input on the operating unit can, with at least high probability, only be carried out by a human. Within the context of the present disclosure, such confirmation can in particular only be carried out by a human being if confirmation by a computer or a machine is not possible or is only possible with considerable effort, the effort being measured, for example, in terms of costs, time and/or technical means. For example, the information in the request message can be generated in accordance with a prompt-response method and/or comprises a puzzle that the user has to solve. The request message does not, for example, comprise a simple request in the form of a confirmation query, where a user simply agrees or disagrees with a configuration change by answering yes or no. Furthermore, it is conceivable that the request message comprises the previously saved configuration value and that this value can be displayed on the operating unit.

The gas measuring device is further configured to receive the response message from the operating unit and to check the information about the manual input from the user that it contains. The information about manual input comprises the user's solution to the task they have to solve in the request message. The test involves comparing the information about the manual input from the user with at least one expected value. It is conceivable that the expected value is predetermined and stored in the gas measuring device and/or that the expected value can be generated by the gas measuring device on the basis of the request message, in particular on the information about a task to be solved.

If the information about the manual input completely or partially deviates from the expected value, or if no response message containing information about manual input is received, it can be assumed that the manual input on the operating unit was not carried out by a human.

The gas measuring device is further configured to generate an information signal and/or switch to the safe state in the event of such a deviation between the information about the manual input and the expected value or the absence of a response message. The information signal comprises, in particular, an indication of a detected deviation or an indication that no response message containing information about manual input has been received. For example, the information signal is a warning signal, an alarm signal and/or an electronic message. The safe state of the gas measuring device depends on the particular protective function that the gas measuring device is required to fulfill. For example, the gas measuring device switches to a special state to indicate that it is not operational. Furthermore, it is conceivable that the gas measuring device might generate an alarm or switch to an error state.

If the information about the manual input substantially matches the at least one expected value, the previously stored configuration value is applied by the gas measuring device, or it remains applied if the configuration value has already been applied previously, in particular after the gas measuring device has received the configuration value.

The particular advantage of the gas measuring device according to the present disclosure lies in the fact that it ensures that a change to a safety-relevant configuration value can only be carried out or confirmed by a human being and not by a computer, a machine or any other electronic unit. Furthermore, it ensures that it is the user who intends to make the change to the configuration value. This allows safe and reliable operation of the gas measuring device.

Furthermore, the present disclosure relates to a method for changing a safety-relevant configuration value of a gas measuring device, comprising the following steps:
sending a configuration value from an operating unit to the gas measuring device,
generating a request message for manual input confirming the configuration value by means of the gas measuring device, wherein the request message comprises information about a task to be solved,
sending the request message to the operating unit,
outputting at least part of the request message by means of the operating unit,
generating a response message on the basis of manual input from a user by means of the operating unit, wherein the manual input relates to the fact that the request message is at least partially output on the operating unit,
sending the response message containing information about the manual input to the gas measuring device,
comparing the information about the manual input with an expected value by means of the gas measuring device,
using the comparison as a basis for generating an information signal by means of the gas measuring device and/or switching of the gas measuring device to a safe state

The method is suitable to be carried out by one of the systems described above or to be at least partially carried out by the gas measuring device described above.

The method according to the present disclosure advantageously allows for reliable and safe changing of a safety-relevant configuration value, whereby it is established whether the configuration change was carried out by a human.

### BRIEF DESCRIPTION OF THE DRAWINGS

In the figures:

FIG. 1 is a schematic representation of an example embodiment of the system according to the present disclosure, and

FIG. 2 is a state diagram of an example embodiment of the gas measuring device according to the present disclosure.

### DETAILED DESCRIPTION

Exemplary embodiments of the present disclosure are described in detail below with reference to the accompanying figures.

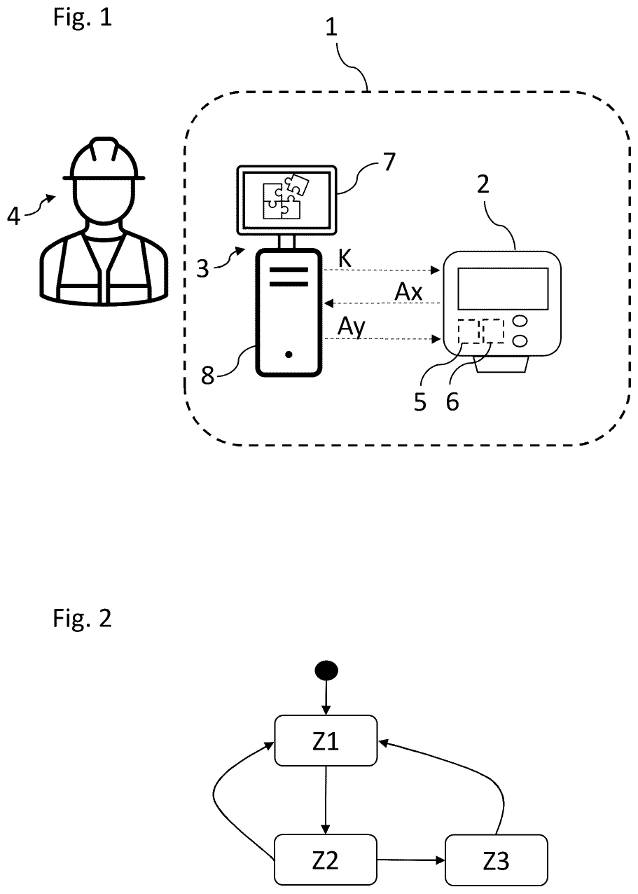

FIG. 1 shows, using a schematic representation, an example embodiment of the system 1 according to the present disclosure. The system 1 comprises a gas measuring device 2 and an operating unit 3, which are connected to one another via a wireless data transmission interface. Furthermore, FIG. 1 shows a user 4 who uses the operating unit to make a change to a safety-relevant configuration value K.

In this example embodiment, the gas measuring device is a stationary gas measuring apparatus, which comprises a computing unit 5 in the form of a microcontroller and a data memory 6 in the form of a semiconductor memory. Furthermore, the gas measuring device 1 comprises a communication unit (not shown) for wireless data transmission and a gas sensor (not shown) for recording a gas in a region around the gas measuring device 1.

The operating unit comprises a touch-sensitive screen 7 and a control unit 8 for controlling the screen 7. Furthermore, the operating unit comprises a communication unit for wireless data transmission.

The user 4 has the option to enter a new configuration value K on the operating unit 3, wherein the operating unit 3 is configured to then transmit the configuration value K to the gas measuring device 2. The configuration value K comprises, for example, a value for an alarm threshold for a gas that can be recorded by the gas sensor of the gas measuring device 2.

The gas measuring device 1 is configured to receive the configuration value K and store it in the data memory 6. Furthermore, the gas measuring device is configured to apply the configuration value K, with the computing unit 5 taking the configuration value K into account when evaluating and/or recording a gas concentration. The value for an alarm threshold comprised in the configuration value K is therefore taken into account from the time the configuration value K is applied for assessing whether an alarm, in particular a dangerous gas concentration, is present.

Furthermore, the gas measuring device 1 is configured to generate a request message Ax to confirm the configuration value K by the user. The request message comprises information about a task to be solved by the user 4 by means of manual input. The task to be solved requires interaction between the user and the operating unit 3. The gas measuring device 1 is configured to transmit the request message to the operating unit.

The operating unit 3 is configured to receive the request message Ax from the gas measuring device 1 and to display at least some of the information contained therein on the screen 7. In this example embodiment, the request message comprises a CAPTCHA in the form of a puzzle, as indicated on the screen 7 in FIG. 1. The puzzle is displayed on the screen 7 and the user 4 is prompted to solve or complete the puzzle. Based on the CAPTCHA to be solved or the puzzle to be completed, it can be determined whether the corresponding task was solved by a human.

The operating unit 3 is further configured to identify manual input from the user 4 to solve the CAPTCHA on the touch-sensitive screen 7 of the operating unit 3 and to generate a response message Ay, wherein the response message Ay contains information about the manual input from the user 4. Furthermore, the operating unit 3 is configured to transmit the response message Ay to the gas measuring device.

The gas measuring device 2 is configured to receive the response message Ay and to check the information about the manual input from the user 4. This involves comparing the information about the manual input from the user 4 with at least one expected value. The expected value and the information about a task to be solved by the user 4 are stored in the data memory 6 of the gas measuring device 2.

In the event that the expected value at least partially deviates from the information about the manual input, for example because the puzzle was not solved or not completely solved, the gas measuring device 1 switches to the safe state, wherein the gas measuring device issues an alarm.

If the expected value matches the information about the manual input, in particular because the puzzle has been completely solved, the gas measuring device continues to operate.

The gas measuring device 1 switches to the safe state, wherein the gas measuring device issues an alarm.

If the information about the manual input completely or partially deviates from the expected value, it may be assumed that the manual input on the operating unit was not carried out by a human.

FIG. 2 shows a state diagram of an example embodiment of the gas measuring device 2 according to the present disclosure with the states Z1, Z2 and Z3 and the respective transitions of the states, represented by arrows. In the first state Z1, the gas measuring device 1 is in operation and records and evaluates gas measurement values.

When the gas measuring device 2 receives a configuration value K, it changes from the first state Z1 to the second state Z2. In this second state Z2, the gas measuring device 2 applies the received configuration value K and takes it into account during continued operation. Furthermore, the gas measuring device 2 is configured in the second state Z2 to generate a request message Ax for manual input and to transmit the request message Ax to an operating unit 3, wherein the request message Ax comprises information about a task to be solved by a user 4. The gas measuring device is further configured to carry out a time measurement from the time the request message Ax was transmitted and to check whether an intended period, for example 15 minutes, has been exceeded.

In the event that the intended period, for example 15 minutes, is not yet up and the gas measuring device receives a response message Ay with information about a manual input, the gas measuring device 2 is further configured to compare the information about the manual input with an expected value. If the information about the manual input matches an expected value, the gas measuring device switches from the second state Z2 back to the state Z1. If the information about the manual input at least partially deviates from an expected value, or if the intended period is up or has been exceeded, the gas measuring device switches from the second state Z2 to the third state Z3.

The gas measuring device is configured to assume a safe state in the third state Z3, wherein the gas measuring device generates an alarm. Furthermore, in the event that the gas measuring device 1 receives a response message Ay with information about a manual input, in the state Z3 the gas measuring device is configured to compare the information about the manual input with an expected value. If the information about the manual input matches an expected value, the gas measuring device switches from the third state Z3 to the state Z1, wherein the previously generated alarm stops are issued on the basis of the safe state of gas measuring device 1. If the information about the manual input at least partially deviates from an expected value, or if the intended period is up, the gas measuring device remains in the third state Z3.

### List of Reference Signs

1 System
2 Gas measuring device
3 Operating unit
4 User
5 Computing unit
6 Data memory
7 Screen
8 Control unit
Z1 First state
Z2 Second state
Z3 Third state

1-9. (canceled)

10. A system for changing a safety-relevant configuration value of a gas measuring device, comprising:
the gas measuring device; and
an operating unit connected to the gas measuring device for bidirectional data transmission,
wherein the operating unit is configured to send a configuration value to the gas measuring device, and wherein the gas measuring device is configured to:
detect one or more of a gas and a gas mixture in a monitoring region of the gas measuring device;
receive the configuration value from the operating unit; and
store the configuration value,
wherein the gas measuring device is further configured to:
generate a request message for manual input confirming the configuration value;
send the request message to the operating unit, the request message comprising information of a task to be solved;
receive a response message comprising information of the manual input from the operating unit; and
based on a comparison between the information of the manual input and an expected value, perform at least one of:
generating an information signal; and
switching to a safe state, and
wherein the operating unit is further configured to:
receive the request message from the gas measuring device;
output at least part of the request message;
generate the response message based on manual input from a user; and
send the response message to the gas measuring device.

11. The system of claim 10, wherein the request message differs from a previous request message, wherein the information of the task to be solved that is comprised in the request message differs from information of a task to be solved that is comprised in the previous request message.

12. The system of claim 10, wherein the gas measuring device is further configured to generate the information signal or switch to the safe state further based on at least one of:
a determination that a predetermined time or a predetermined time period between receiving the configuration value and receiving the response message is exceeded; and
a determination that the information of the manual input does not match the expected value within the predetermined time or the predetermined time period.

13. The system of claim 10, wherein the request message comprises a fully automated public Turing test for distinguishing between computers and humans (CAPTCHA).

14. The system of claim 10, wherein the bidirectional data transmission between the gas measuring device and the operating unit is based on:
a Highway Addressable Remote Transducer (HART) communication protocol,
an Advanced Physical Layer (APL) standard, or
Bluetooth.

15. The system of claim 10, wherein the operating unit is further configured to:
authenticate a user; and
send the configuration value to the gas measuring device only after the user has been successfully authenticated.

16. The system of claim 10, wherein the gas measuring device possesses the properties required in the standard IEC 61508 and is configured as a functional safety electronic device.

17. A gas measuring device for changing a safety-relevant configuration value, wherein the gas measuring device is configured to:
detect at least one of a gas or a gas mixture in a monitoring region of the gas measuring device;
receive a configuration value from an operating unit;
store the configuration value;
generate a request message for manual input confirming the configuration value;
send the request message to the operating unit, wherein the request message comprises information of a task to be solved
receive a response message with information of the manual input from the operating unit; and
based on a comparison between the information of the manual input and an expected value, perform at least one of:
generating an information signal; and
switching to a safe state.

18. The gas measuring device of claim 17, wherein the gas measuring device is further configured to generate the information signal or switch to the safe state further based on at least one of:
a determination that a predetermined time or a predetermined time period between receiving the configuration value and receiving the response message is exceeded; and
a determination that the information of the manual input does not match the expected value within the predetermined time or the predetermined time period.

19. The gas measuring device of claim 17, wherein the request message comprises a fully automated public Turing test for distinguishing between computers and humans (CAPTCHA).

20. The gas measuring device of claim 17, wherein the gas measuring device is further configured for bidirectional data transmission between the gas measuring device and the operating unit based on:
a Highway Addressable Remote Transducer (HART) communication protocol,
an Advanced Physical Layer (APL) standard, or
Bluetooth.

21. The gas measuring device of claim 17, wherein the gas measuring device possesses the properties required in the standard IEC 61508 and is configured as a functional safety electronic device.

22. A method for changing a safety-relevant configuration value of a gas measuring device, comprising the steps of:
sending a configuration value from an operating unit to the gas measuring device;
generating a request message for manual input confirming the configuration value from the gas measuring device, wherein the request message comprises information of a task to be solved;
sending the request message to the operating unit;
outputting, by the operating unit, at least part of the request message;
generating, by the operating unit, a response message based on manual input from a user;
sending the response message to the gas measuring device, wherein the response message comprises information of the manual input;
comparing, by the gas measuring device, the information of the manual input with an expected value;
performing, based on the comparison, at least one of:
generating an information signal; and
switching the gas measuring device to a safe state.

23. The method of claim 22, wherein the request message differs from a previous request message, wherein the information of the task to be solved that is comprised in the request message differs from information of a task to be solved that is comprised in the previous request message.

24. The method of claim 22, wherein the request message comprises a fully automated public Turing test for distinguishing between computers and humans (CAPTCHA).

25. The method of claim 22, wherein the sending the request message and the sending the configuration value are based on:
a Highway Addressable Remote Transducer (HART) communication protocol,
an Advanced Physical Layer (APL) standard, or
Bluetooth.

26. The method of claim 22, further comprising:
authenticating, by the operating unit, a user; and
wherein the sending the configuration value is performed only after the user has been successfully authenticated.

27. The method of claim 22, wherein the gas measuring device possesses the properties required in the standard IEC 61508 and is configured as a functional safety electronic device.
